(19) United States
(12) Patent Application Publication
(10) Pub. No.: US 2010/0191299 A1
(43) Pub. Date: Jul. 29, 2010

(54) Electrical Contact of Biocompatible Material

(76) Inventor: Mark Ayzenberg, Hudson, NH (US)

Correspondence Address: BRAKE HUGHES BELLERMANN LLP, c/o CPA Global, P.O. BOX 52050, MINNEAPOLIS, MN, 55402, US

(21) Appl. No.: 12/360,848

(22) Filed: Jan. 27, 2009

(51) Int. Cl. A61N 1/375; H01R 13/187; H01R 13/10

(52) U.S. Cl. 607/2; 439/843; 439/682

(57) ABSTRACT

An electrical contact of biocompatible material for providing an electrical connection between an implantable medical device and an electrical lead component is provided. The contact including a ferrule of biocompatible material having a ridge with a diameter being greater than a body diameter of the ferrule. Conducting wires may be strung within the ferrule positioned at an angle to a longitudinal axis of the ferrule to form a hyperboloid wire cage within the ferrule, the conducting wires comprising a biocompatible material. The wire cage may have an inner diameter that is smaller than the outer lead diameter, such that upon insertion of the electrical lead component into the hyperboloid wire cage, the conducting wires tension around the outer lead diameter of the lead component. A casing member, of biocompatible material, may be arranged to fit securely around a ridge of the ferrule, arranged to enclose the ferrule.

### TECHNICAL FIELD

This description relates to electrical contacts of biocompatible material.

### BACKGROUND

Electrical contacts provide a physical interface through which two devices or components may establish an electrical connection. For example, in the field of implantable medical devices, a contact may provide the physical interface between a surgically implantable medical device and one or more electrical leads that contact a body cavity in which the device is being implanted. The contact may be molded into or installed within the medical device, and then during the surgical procedure one or more electrical leads may be positioned within the body cavity and connected to the medical device via the contact.

### SUMMARY

According to an example embodiment, an electrical contact of biocompatible material for providing an electrical connection between an implantable medical device and an electrical lead component having an outer lead diameter is provided. A ferrule may have a body diameter associated with a body portion of the ferrule and a ridge diameter associated with a ridge of the ferrule, the ridge diameter being greater than the body diameter, the ferrule comprising a biocompatible material. A plurality of conducting wires may be strung within the ferrule from a first end to a second end of the ferrule and positioned at an angle to a longitudinal axis of the ferrule to form a hyperboloid wire cage within the ferrule, the conducting wires comprising a biocompatible material, wherein the wire cage has an inner diameter that is smaller than the outer lead diameter, such that upon insertion of the electrical lead component into the hyperboloid wire cage, the conducting wires tension around the outer lead diameter of the lead component. A first casing member and a second casing member, at least one of which may be arranged to fit securely around a ridge of the ferrule, arranged to enclose the ferrule wherein the first casing member contacts the second casing member, and wherein the first and second casing members comprise a biocompatible material

According to an example embodiment a system is provided. A lead component may be adapted to receive sensory inputs from within a living body. An implantable medical device may be configured to receive the sensory inputs and provide electrical impulses, based on the sensory inputs, to the living body via the lead component. A electrical contact of biocompatible material may be arranged to form an electrical connection between the lead component and the implantable medical device upon insertion of the lead component into a hyperboloid wire cage of the electrical contact of biocompatible material, wherein the hyperboloid wire cage may be arranged to expand upon the insertion of the lead component to form the electrical connection via a plurality of conducting wires comprising a biocompatible material and transmit the sensory inputs and electrical impulses between the lead component and implantable medical device. A casing member, comprising a biocompatible material may be adapted to contact the living body, of the electrical contact of biocompatible material may be arranged to enclose the hyperboloid wire cage, the casing member having a receiving end for receiving the lead component and a connection end configured for connectivity with the implantable medical device.

According to an example embodiment a female electrical contact for providing an electrical connection between an implantable medical device and a male lead component having an outer lead diameter, wherein the female electrical contact is arranged to be molded to the implantable medical device is provided. A plurality of conducting wires comprising biocompatible material may be positioned at an angle to a longitudinal axis of the female electrical contact to form a hyperboloid wire cage, the wire cage having a relaxed inner cage diameter that may be less than the outer lead diameter. The hyperboloid wire cage may be arranged to mate with the male lead component to form the electrical connection between the male lead component and the implantable medical device, the plurality of conducting wires configured to tension around the male lead component, such that the wire cage may have a tensioned inner diameter greater than the relaxed inner diameter, upon insertion of the male lead component into the hyperboloid wire cage to form multiple electrical contact paths between the conducting wires and male lead component. A casing member may be arranged to enclose the hyperboloid wire cage, wherein the casing member comprises a biocompatible material adapted to contact fluid and/or tissue from a living body when the casing member and the portion of the male lead component are located within the living body. An elastomeric sealing member may be arranged within the casing member to seal the hyperboloid wire cage from contact with the implantable medical device.

The details of one or more implementations are set forth in the accompanying drawings and the description below. Other features will be apparent from the description and drawings, and from the claims.

### BRIEF DESCRIPTION OF THE DRAWINGS

FIG. 1 is an exploded isometric view of an example electrical contact of biocompatible material.

FIG. 2 is an isometric end view of an example electrical contact of biocompatible material.

FIG. 3 is a cross-sectional view of the electrical contact of biocompatible material of FIG. 2.

FIG. 4 is a cross-sectional cut out view of the electrical contact of biocompatible material of FIG. 2.

FIG. 5A is a side view of an implantable medical device that includes an electrical contact of biocompatible material, according to an example embodiment.

FIG. 5B is a side view of an implantable medical device that includes an electrical contact of biocompatible material and an electrical lead inserted into the contact, according to an example embodiment.

### DETAILED DESCRIPTION

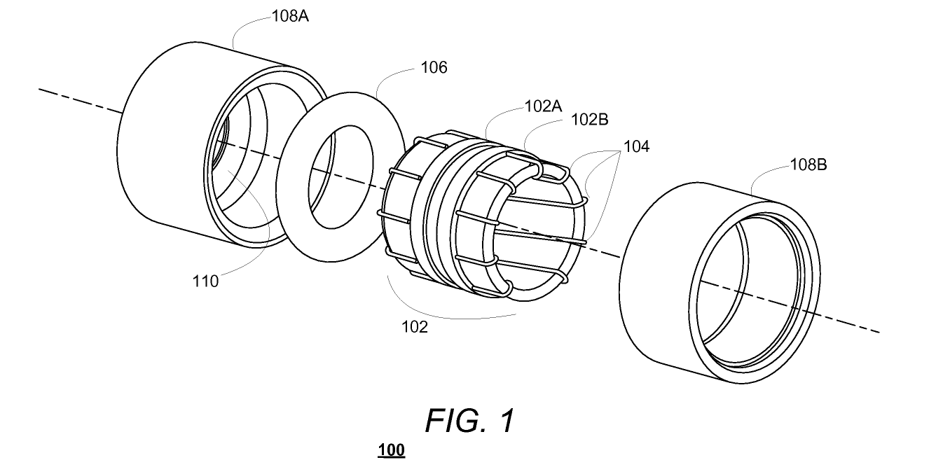

FIG. 1 is an exploded isometric view of an example electrical contact of biocompatible material 100. The electrical contact of biocompatible material 100 may provide a conductive medium or physical interface for electrically and physically coupling electrical components together. The electrical contact of biocompatible material 100 may be used, for example, in surgical procedures to provide a physical interface between an implantable medical device and an electrical lead component within a body cavity, such as a human body, animal body or other living body. The physical interface may provide for an electrical connection, or for current to be passed, between the medical device and the lead component via the electrical contact of biocompatible material 100. For example, the electrical contact of biocompatible material 100 may transmit sensory inputs received by the lead component from the body cavity to the medical device, and/or electrical impulses from the medical device to the lead component, which may then be applied to the body cavity by the lead component.

In the example of FIG. 1, the electrical contact of biocompatible material 100 includes a ferrule 102. The ferrule 102 may include a cylindrical sleeve, or body, with an opening at each end. The ferrule 102 may house multiple conducting wires 104 that may be positioned to form a hyperboloid wire cage within the ferrule 102. The hyperboloid wire cage may receive or mate with the electrical lead component (not shown) to form the physical interface or electrical connection as discussed above. For example, the hyperboloid wire cage may include a female wire cage that may be arranged to mate with a male lead component to form or establish the physical interface over which electrical current may be passed.

In an example embodiment, the electrical contact of biocompatible material 100 may include an elastomeric sealing member 106. The elastomeric sealing member 106 may fit inside an inner groove 110 within a first casing member 108A to prevent foreign substances from contacting the ferrule 102 including the conducting wires 104. For example, during a molding process in which the electrical contact of biocompatible material 100 may be molded to an implantable medical device (not shown), the elastomeric sealing member 106 may prevent molding material from contacting the ferrule 102, including the conducting wires 104. The first casing member 108A, including the elastomeric sealing member 106 positioned within the inner groove 110, and a second casing member 108B may be press-fit together to encase the ferrule 102 including the hyperboloid wire cage formed by the conducting wires 104.

As just referenced, the ferrule 102 may include a body, cylindrical sleeve, ring or cage upon which the conducting wires 104 may be strung. The ferrule 102 may provide housing or other structure for the hyperboloid wire cage. For example, the conducting wires 104 may be strung from a first end or opening of the ferrule 102, across the inner body of the ferrule 102 forming the hyperboloid wire cage, to a second end or opening of the ferrule 102.

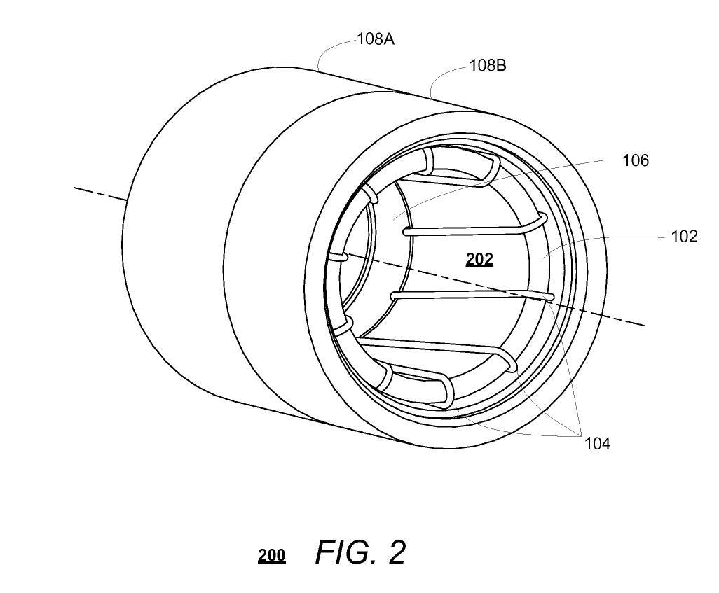

An example hyperboloid wire cage 202, as formed within the ferrule 102, may be seen more clearly in FIG. 2. As may be seen in FIG. 2, the hyperboloid wire cage 202 may be formed from multiple conducting wires 104 extending through the ferrule 102 at an angle to the central axis of the ferrule (as represented by the dashed line). For example, the conducting wires 104 may be positioned or strung through the inner bore of the ferrule 102, with the conducting wires 104 extending beyond the end of the ferrule 102 and wrapping or bending around the end onto the outer surface of the ferrule 102, as illustrated in FIG. 1 and in FIG. 2.

The hyperboloid wire cage 202 may provide the cavity or receptacle where an electrical component (not shown) may be inserted into or otherwise mated with the electrical contact of biocompatible material 100, to establish an electrical connection. The electrical connection may be established when, for example, the conducting wires 104 of the hyperboloid wire cage 202 make physical contact with the electrical lead component. According to an example embodiment, the electrical connection between an implantable medical device, which may include the electrical contact of biocompatible material 102, and one or more lead components may result from establishing a physical interface or connection between the electrical contact 102 and the lead component.

The physical interface between the electrical contact of biocompatible material 102 and the lead component may be established during a mating procedure in which a male lead component may be inserted into the female contact 102. One skilled in the art may appreciate that during the mating procedure a lower insertion force may be preferred such that the mating procedure may be more easily performed (e.g., by a surgeon or other technician implanting the implantable medical device within the body cavity) and also reducing the risk of possible damage to the electrical lead and/or conducting wires 104.

The amount of insertion force required during the mating procedure may be correlated to the length of the conducting wires 104, the number of conducting wires 104 and the angles at which the conducting wires 104 may be strung within the ferrule 102 to form the wire cage 202. For example, shorter conducting wires 104, a greater number of conducting wires 104 and a greater angular variance at which the conducting wires 104 are strung to form the hyperboloid wire cage 202 may result in a greater required insertion force during the mating procedure and vise verse.

One of the challenges overcome with the electrical contact of biocompatible material 102 of FIG. 1, is that, despite that longer conducting wires (e.g., 104) allow for a greater and thus more reliable surface of the conducting wires available for the electrical connection between the lead component and medical device, and generally reduced insertion force during mating, the conducting wires 104 of the electrical contact of biocompatible material 102 may be shortened to address the space constraints of modern implantable medical devices, arranged with the path of insertion of the lead component into the electrical contact of biocompatible material 102 (including, for example, being strung at a slight angular variance to the path of insertion to form the wire cage), and still maintaining a low insertion force during mating to reduce the likelihood of damage. The conducting wires 104 may be arranged to form the wire cage 202 such that a relatively low insertion force may be used to insert or otherwise mate the electrical component with the wire cage 202 to form the physical and electrical connections.

Thus, the dimensions of the electrical contact of biocompatible material 100 may be selected such that the length of the body of the ferrule 102 and the conducting wires 104 is relatively short, while nevertheless achieving a low insertion force to establish a physical interface with an electrical component and high reliability of an electrical connection between the electrical component and the electrical contact of biocompatible material 102. For example, according to non-limiting example embodiments, the length of the electrical contact of biocompatible material 100 may be as substantially equal to about 10 mm, 8, mm, 6 mm, 4 mm, or 2 mm, and the length of the conducting wires 104 from one end of the ferrule 102, and the diameter of the wire cage 202 formed by the conducting wires 104 can be substantially equal to about 3.0 mm, 2.5 mm, 2.0 mm, or 1.5 mm. According to another example embodiment, a ratio of the length of the wire cage 202 to the diameter of the wire cage 202 may be less than 2.

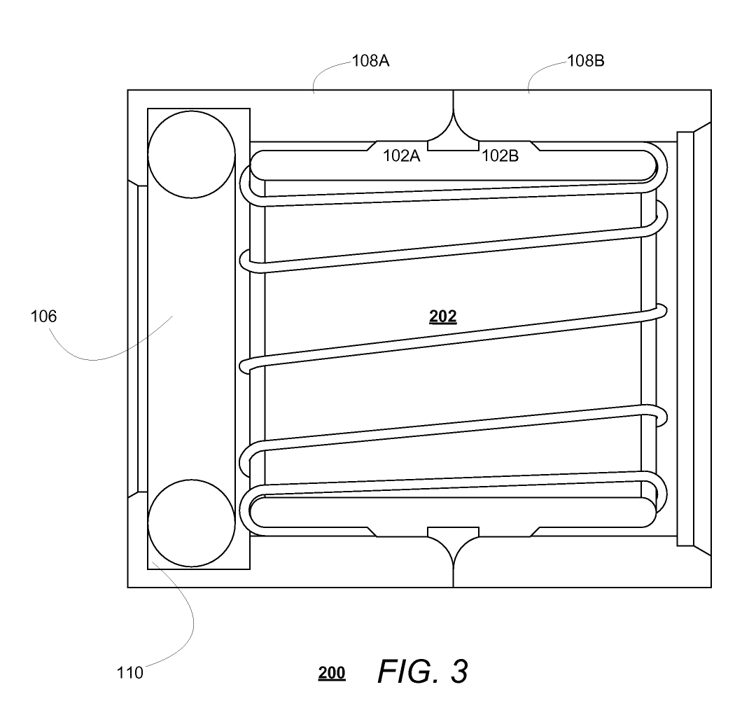

As shown in FIG. 3, within the ferrule 102, the conducting wires 104 may be positioned at an angle or angular variance to a longitudinal axis of the ferrule 102 to form the wire cage 202. Thus, in the section plane shown in FIG. 3, the ends of the conducting wires 104 extending through, beyond or over the right side of the ferrule 102 (e.g., on the casing member 108B side) appear slightly higher than those on the left side (e.g., on the casing member 108A side) due to this angular variance. The angle or angular variance of the conducting wires 104 may vary based upon the application of the electrical contact of biocompatible material 200. The electrical contact of biocompatible material 200 may be substantially similar to the electrical contact of biocompatible material 100, except that the electrical contact of biocompatible material 100 is shown as an exploded isometric view and the electrical contact of biocompatible material 200 is shown as an assembled isometric view.

The angular variance of the conducting wires 104 may cause the conducting wires 104 to be suspended above an inner surface of the ferrule 102. The greater the angle at which a conducting wire 104 is strung within or across the ferrule 102, the greater the height of suspension above the inner surface of the ferrule 102. This suspension above the inner surface of the ferrule 102 may result in forming the wire cage 202 as discussed above. The distance of the conducting wire 104 to the inner surface of the ferrule 102 as a function of distance from one end of the ferrule 102 to the other may approximate a hyperboloid function. Thus, the diameter of the wire cage 202 may be smallest at the midpoint of the distance between the two ends of the ferrule 102 and greatest at the ends of the ferrule 102.

A male electrical lead component may be inserted into the female wire cage 202, which may cause the female wire cage 202 (e.g., conducting wires 104) to mate with the male lead component and expand to the outer diameter of the lead component creating the physical interface. While the angle of the conducting wires 104 may vary, the conducting wires 104 should be strung at such an angle so as to avoid undue tension on the conducting wires 104 upon insertion of the electrical lead component to the wire cage 202, as discussed above.

Also as referenced above, another cause of tension on the conducting wires 104 may result from shortening the length of the conducting wires 104 to accommodate a shorter ferrule 102 adapted for implantation into a human body or other body cavity. Shorter conducting wires 104 may result in reduced angles at which the conducting wires 104 may be strung inside the ferrule 102 and/or greater tension in a relaxed state thus resulting in less allowed tension upon insertion of the male lead component. The electrical contact of biocompatible material 100, 200 may overcome such challenges by balancing the length of the conducting wires 102 with the angular variance to accommodate for a low insertion force.

One or more electrical components within the implantable medical device may be electrically connected to the conducting wires 104. The conducting wires 104 may include biocompatible wires that can transmit an electrical signal between a lead component and the electrical component(s) of the implantable medical device. The conducting wires 104 may each provide a contact path by which the medical device and lead component may communicate. An advantage of having various distinct contact paths may be that if one or more of the conducting wires 104 is damaged, the remaining conducting wires 104 may be able to maintain the electrical connection. For example, if one of the conducting wires 104 breaks, then the remaining unbroken or undamaged wires may maintain the electrical connection between the electrical lead component and implantable medical device. This may be advantageous in implantable medical devices where failure of the electrical contact of biocompatible material 100 may result in serious injury or death.

As shown in the example of FIG. 1, the conducting wires 104 on the outside surface of the ferrule 102 may extend towards one or more ridges 102A, 102B of the ferrule 102. The ferrule 102 may include one or more ridges 102A, 102B that may include a raised edge or surface on the outer surface of the ferrule 102. The ridges 102A, 102B may be of a height, or ridge diameter, approximately equal to or slightly greater than the inner diameter of the casing members 108A and 108B, such that one or more of the casing members 108A and 108B that may be press-fit onto or over the ridges 102A, 102B of the ferrule 102. According to an example embodiment, the ridge diameter may be greater than the body diameter of the ferrule 102 by an amount approximately equal to or slightly greater than twice the diameter of the conducting wires 104 pressed onto the outer surfaces of the body of the ferrule 102. Or put another way, the diameter of a conducting wire 104 may be equal to one-half the difference between the ridge diameter the body diameter of the ferrule 102. Then, for example, the casing members 108A, 108B may secure the conducting wires 104 to the outer surface of the ferrule 102 when press-fit onto the ridges 102A, 102B, without applying excessive force to the conducting wires 104. As referenced above, in an example embodiment, the ferrule 102 may include a single ridge 102A, or perhaps multiple ridges 102A, 102B beyond the two shown in the example of FIG. 1, such that the casing members 108A, 108B may be press-fit onto one or more of the ridges 102A, 102B.

Referring to FIG. 1, the inner groove 110 may be arranged to fit or hold the elastomeric sealing member 106. The inner groove 110 may be included on the interior of one or both of the casing members 108A and 108B, in which the interior diameter of the inner groove 110 may be greater than the remainder interior diameter of the casing member 108A, 108B. The inner groove 110 may serve to house or hold the elastomeric sealing member 106 in position when the biocompatible electric contact 100 is assembled. The inner groove 110 may prevent the elastomeric sealing member 106 from moving around within the biocompatible electric contact 100. Though only one elastomeric sealing member 106 and inner groove 110 is shown in FIG. 1, other example embodiments may include no elastomeric sealing member 106 or inner groove 110 or multiple elastomeric sealing members 106 and inner grooves 110. Though designed primarily to house the elastomeric sealing member 106, other example embodiments without the elastomeric sealing member 106 may still include the casing member 108A with the inner groove 110.

The elastomeric sealing member 106 may be placed or otherwise fit into the inner groove 110 and, in cooperation with an insertion member (not shown) placed within the sealing member, can prevent molding material or other foreign substances from entering the inside of the electrical contact 100. Placed within the inner groove 110, the elastomeric sealing member 106 may prevent silicon, rubber or other liquids or substances from contacting to ferrule 102 or the conducting wires 104 during a molding or over-molding process during which the electrical contact of biocompatible material 100 may be molded into an implantable medical device. According to an example embodiment, a longitudinal insertion member (not shown) may be inserted into the biocompatible contact and may conjoin with the elastomeric sealing member 106 to seal off the inside of the ferrule 102 from making contact with the molding substance. Then for example, the longitudinal insertion member may be removed when the electrical contact 102 has been molded within the implantable medical device and the molding process is complete.

The ferrule 102, the conducting wires 104, the elastomeric sealing member 106 and the casing members 108A, 108B each may include or may otherwise be composed of a biocompatible material. One or more of the components may be composed of similar biocompatible material or different components may be composed of different biocompatible materials. The biocompatible material may include any material that may be safely implanted or arranged within a body cavity. For example, the biocompatible materials may include any material that is approved for usage within a body cavity, including a human body, by an organization that may be governmental, industry organized or otherwise collaborative. Exemplary organizations may include the Food and Drug Administration (FDA), American Medical Association (AMA) or the European Society for Biomaterials.

The biocompatible materials however may not have been certified safe by any particular organization, but may have been tested and/or otherwise deemed safe for usage with or within a body cavity. For example, the biocompatible material should not harm the body upon making contact with tissue, fluids and/or other chemicals of the body cavity while making contact with human tissue. Example biocompatible materials may include, but are not limited to, silicone based compounds, such as those made by the NuSil Technology LLC of Carpinteria, Calif. (e.g., NuSil MED4850 or NuSil MED4870) that may be used with the elastomeric sealing member 106, non-reactive metal alloys, such as, for example, platinum/iridium allows (e.g., PT-20% IR alloy) that may be used with the conducting wires 104, and alloys of nickel, cobalt, chromium, molybdenum (e.g., MP35N) may be used with the ferrule 102 and the casing members 108A and 108B. In other example embodiments, other various biocompatible materials may be used with the electrical contact of biocompatible material 100.

The body cavity may include any organic body cavity. For example, the body cavity may include any portion of a human or animal body cavity, including extremities. The body cavity may include muscle, tissue, fat, blood, mucus and/or other liquids or substances. The biocompatible material should not cause harm when in contact with any of the substances of the body cavity in which the implantable medical device may be placed. Also, according to an example embodiment, the substances of the body cavity ideally would not cause the biocompatible material to rust, corrode or otherwise degrade.

FIG. 2 is an isometric end view of an example electrical contact of biocompatible material 200. The electrical contact of biocompatible material 200 may include the components of the electrical contact of biocompatible material 100 as assembled into a unit.

In the electrical contact of biocompatible material 200, the hyperboloid wire cage 202 may be seen within the ferrule 102. The hyperboloid wire cage 202 may include multiple conducting wires 104 as positioned or otherwise strung on or across the ferrule 102. For example, in the example of FIG. 2, the wire cage 202 may include 10 conducting wires 104 positioned uniformly around the circumference of the ferrule 102. Other example embodiments may include more or fewer conducting wires 104 placed at uniform or non-uniform distances around the circumference of the ferrule 102. As referenced above, the number of conducting wires 104, their angular variance and where they are placed along the ferrule 102 may be adjusted to account for maintaining a low insertion force during a mating process with an electrical lead component.

FIG. 3 is a cross-sectional view of the electrical contact of biocompatible material 200 of FIG. 2. In the cross-sectional view of FIG. 3, it may be seen how the casing member 108B may be press-fit around the ridge 102B of the ferrule 102 and may secure one end of the conducting wires 104. Similarly the casing member 108A may be press-fit around the ridge 102A of the ferrule and secure the other ends of the conducting wires 104. As discussed above, the cross-section of the ferrule 102 shows the angular variance among the conducting wires 104 positioned along the longitudinal axis of the ferrule 102 to form the hyperboloid wire cage 202.

In the example electrical contact of biocompatible material 200, the left side (e.g., casing member 108A side) of the contact 200 may be molded into or otherwise with an implantable medical device. In such a case, the elastomeric sealing member 106 may prevent any substance or molding to seep into the remainder of the electrical contact of biocompatible material 200. Then for example, an electrical lead component may be inserted into the right side (e.g., the casing member 108B side) of the contact 200 and be secured within the wire cage 202. In other example embodiments, the electrical lead component may be inserted through the other side (e.g., casing member 108A side) or either side of the contact 200.

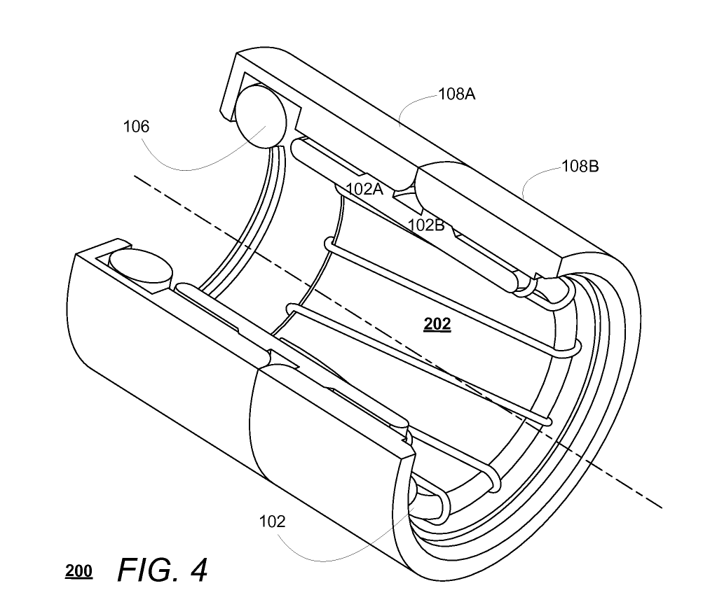

FIG. 4 is a cross-sectional cut out view of the electrical contact of biocompatible material 200 of FIG. 2. As may be seen in FIG. 4, the ridges 102A and 102B may extend around the outer circumference of the ferrule 102 and be pressed against the inner circumference of the casing members 108A and 108B. Also as may be seen, the casing members 108A and 108B may be pressed firmly against each other forming a housing or encasement around the ferrule 102 and conducting wires 104. In other example embodiments, additional casing members 108A, 108B may be used to form the housing or casement of the contact 200.

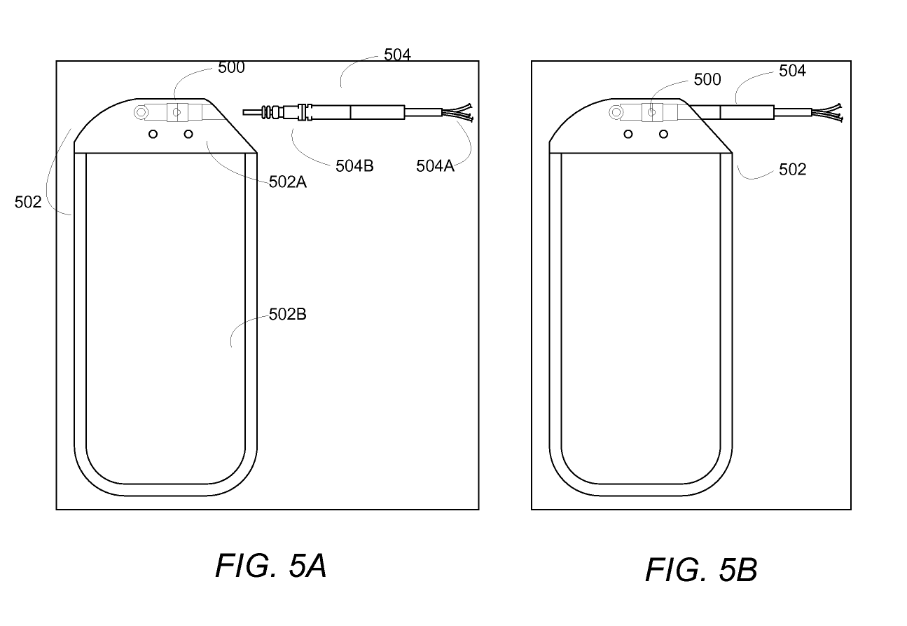

FIG. 5A is a side view of an implantable medical device 502 that includes an electrical contact of biocompatible material 500, according to an example embodiment. As referenced above, the contact 500 may be molded to the implantable medical device 502. Then for example, an electrical lead 504 may be inserted into the implantable medical device 502 through the contact 500.

The implantable medical device 502 may include any medical device useful for diagnostic or therapeutic purposes. For example, the implantable medical device 502 may be used for the diagnosis, monitoring, treatment and/or alleviation of any disease, injury or other ailment. Example implantable medical devices 502 may include, but not be limited to, pacemakers, ICDs (implantable cardioverter-defibrillators), neurostimulators, metabolic controls, circulation pumps, bone growth stimulators and pain management devices. As referenced above, the implantable medical device 502, with the installed or molded electrical contact 500, may be implantable or otherwise arranged within a body cavity.

The implantable medical device 502 may include a connection portion 502A and a device portion 502B. The connection portion 502A, as indicated in FIG. 5A, may include the contact 500 molded into or otherwise with the connection portion 502A. The connection portion 502A may be configured to receive and establish a connection with the electrical lead 504 via the contact 500. The device portion 502B may include the circuitry of the implantable medical device 502 hermetically sealed inside the shell of the device 502. According to a non-limiting example embodiment, the device portion 502B may be sealed inside a biocompatible container that may, for example, be made of titanium. In other example embodiments, other biocompatible materials other than titanium may be used.

The electrical lead 504 may include a device for establishing an electrical pathway between a body cavity and the implantable medical device 502. The electrical lead 504 may include cylindrical or prong-like component adapted to connect with the implantable medical device 502.

The electrical lead 504 may include a sensor portion 504A and a contact area portion 504B. The sensor portion 504A may include one or more sensors, antenna, wires, or other feelers configured to contact a body cavity. The sensor portion 504A may receive sensory inputs from the body cavity which may be transmitted through the electrical lead 504 to the contact area portion 504B. The contact area portion 504B may include a male contact area arranged to make contact and/or otherwise mate with the female electrical contact of biocompatible material 500 molded with the implantable medical device 502. As discussed above, the contact portion 504B may have an outer diameter greater than the inner diameter of the wire cage (e.g., 202) of the contact 500, such that the conducting wires (e.g., 104) stretch around the contact portion 504B creating the physical interface between the electrical lead 504 and the implantable medical device 502. Then for example, via the physical interface created by the contact 500, the sensory inputs received by the sensor portion 504A may be transmitted to the device portion 502B of the medical device 502. Upon processing of the sensory inputs, the medical device 502 may then indicate or provide electrical stimulation via the physical interface (as formed by the contact 500) to the electrical lead 504 to be applied to the body cavity via the sensor portion 504A.

FIG. 5B is a side view of an implantable medical device 502 that includes an electrical contact of biocompatible material and an electrical lead 504 inserted into the contact, according to an example embodiment. As shown in FIG. 5B, the electrical lead 504 is inserted into the implantable medical device 502 and a physical interface, and potential electrical interface, may be established via the molded electrical contact of biocompatible material 500.

While certain features of the described implementations have been illustrated as described herein, many modifications, substitutions, changes and equivalents will now occur to those skilled in the art. It is, therefore, to be understood that the appended claims are intended to cover all such modifications and changes as fall within the true spirit of the embodiments.

What is claimed is:

1. An electrical contact of biocompatible material for providing an electrical connection between an implantable medical device and an electrical lead component having an outer lead diameter, the electrical contact of biocompatible material comprising:
a ferrule having a body diameter associated with a body portion of the ferrule and a ridge diameter associated with a ridge of the ferrule, the ridge diameter being greater than the body diameter, the ferrule comprising a biocompatible material;
a plurality of conducting wires strung within the ferrule from a first end to a second end of the ferrule and positioned at an angle to a longitudinal axis of the ferrule to form a hyperboloid wire cage within the ferrule, the conducting wires comprising a biocompatible material, wherein the wire cage has an inner diameter that is smaller than the outer lead diameter, such that upon insertion of the electrical lead component into the hyperboloid wire cage, the conducting wires tension around the outer lead diameter of the lead component; and
a first casing member and a second casing member, at least one of which being arranged to fit securely around a ridge of the ferrule, arranged to enclose the ferrule wherein the first casing member contacts the second casing member, and wherein the first and second casing members comprise a biocompatible material.

2. The electrical contact of biocompatible material of claim 1 wherein the conducting wires are suspended above an inside surface of the ferrule, forming the hyperboloid wire cage.

3. The electrical contact of biocompatible material of claim 1 wherein the ferrule includes a first end opening and a second end opening and wherein the conducing wires extend through, and bend around, the first end opening and the second end opening and contact an outer surface of the ferrule.

4. The electrical contact of biocompatible material of claim 3 wherein a diameter of the conducting wires is substantially equal to half the difference between the ridge diameter and the body diameter of the ferrule.

5. The electrical contact of biocompatible material of claim 1 wherein the biocompatible materials of the ferrule, the conducting wires, and the casing members include material that is approved for use in a human-implantable medical device.

6. The electrical contact of biocompatible material of claim 1 wherein the first casing member further comprises a circumferential inner groove with an inner groove diameter greater than an inner body diameter of the first casing member.

7. The electrical contact of biocompatible material of claim 6 further comprising an elastomeric sealing member arranged to fit in the inner groove.

8. The electrical contact of biocompatible material of claim 7 wherein the elastomeric sealing member, when positioned in the inner groove, is arranged to cooperate with a longitudinal insertion member to prevent a foreign substance from entering the hyperboloid wire cage during a molding process in which the electrical contact of biocompatible material is molded to the implantable medical device.

9. The electrical contact of biocompatible material of claim 1 wherein an inner diameter of the first casing member is arranged to mate with the ridge via a press-fit coupling.

10. The electrical contact of biocompatible material of claim 9 wherein the ferrule includes a second ridge and wherein an inner diameter of the second casing member is arranged to mate with the second ridge via a press-fit coupling.

11. The electrical contact of biocompatible material of claim 1 wherein a ratio of a length of the wire cage to a diameter of the wire cage less than 2.

12. The electrical contact of biocompatible material of claim 1 wherein the conducting wires are arranged at a length such that reducing the length of the conducting wires would result in increasing an insertion force associated with the insertion of the male lead component into the hyperboloid wire cage such that the conducting wires are unable tension around the outer lead diameter of the male lead component at the reduced length.

13. A system comprising:
a lead component adapted to receive sensory inputs from within a living body;
an implantable medical device configured to receive the sensory inputs and provide electrical impulses, based on the sensory inputs, to the living body via the lead component;
an electrical contact of biocompatible material arranged to form an electrical connection between the lead component and the implantable medical device upon insertion of the lead component into a hyperboloid wire cage of the electrical contact of biocompatible material,
wherein the hyperboloid wire cage is arranged to expand upon the insertion of the lead component to form the electrical connection via a plurality of conducting wires comprising a biocompatible material and transmit the sensory inputs and electrical impulses between the lead component and implantable medical device; and
a casing member, comprising a biocompatible material adapted to contact the living body, of the electrical contact of biocompatible material arranged to enclose the hyperboloid wire cage, the casing member having a receiving end for receiving the lead component and a connection end configured for connectivity with the implantable medical device.

14. The system of claim 13 wherein the electrical contact of biocompatible material is arranged to be molded to the implantable medical device.

15. The system of claim 14 wherein the electrical contact of biocompatible material further comprises an elastomeric sealing member arranged within the casing member to seal the hyperboloid wire cage from contacting foreign substances.

16. A female electrical contact for providing an electrical connection between an implantable medical device and a male lead component having an outer lead diameter, wherein the female electrical contact is arranged to be molded to the implantable medical device, the female electrical contact comprising:
a plurality of conducting wires comprising biocompatible material positioned at an angle to a longitudinal axis of the female electrical contact to form a hyperboloid wire cage, the wire cage having a relaxed inner cage diameter that is less than the outer lead diameter;
the hyperboloid wire cage arranged to mate with the male lead component to form the electrical connection between the male lead component and the implantable medical device, the plurality of conducting wires configured to tension around the male lead component, such that the wire cage has a tensioned inner diameter greater than the relaxed inner diameter, upon insertion of the male lead component into the hyperboloid wire cage to form multiple electrical contact paths between the conducting wires and male lead component;
a casing member arranged to enclose the hyperboloid wire cage, wherein the casing member comprises a biocompatible material adapted to contact fluid and/or tissue from a living body when the casing member and the portion of the male lead component are located within the living body; and
an elastomeric sealing member arranged within the casing member to seal the hyperboloid wire cage from contact with the implantable medical device.

17. The female electrical contact of claim 16 wherein the hyperboloid wire cage is arranged such that if one or more of the conducting wires is damaged while tensioned around the male lead component, any remaining unbroken conducting wires maintain their associated electrical contact paths.

18. The female electrical contact of claim 16 wherein the hyperboloid wire cage is arranged within a ferrule of the female electrical contact, the conducting wires being secured to the ferrule by the casing member.

19. The female electrical contact of claim 16 wherein the casing member is press-fit around the hyperboloid wire cage.

20. The female electrical contact of claim 16 wherein the plurality of conducting wires are strung uniformly around a circumference of a ferrule.
